(19) United States
(12) Patent Application Publication
(10) Pub. No.: US 2010/0330684 A1
(43) Pub. Date: Dec. 30, 2010

(54) DIAGNOSTIC DEVICE AND METHOD

(75) Inventor: Stephen D. O'Connor, Leawood, KS (US)

Correspondence Address: INTELLECTUAL PROPERTY / TECHNOLOGY LAW, PO BOX 14329, RESEARCH TRIANGLE PARK, NC, 27709, US

(73) Assignee: HydraDX, Inc., Pasadena, CA (US)

(21) Appl. No.: 12/881,166

(22) Filed: Sep. 13, 2010

Related U.S. Application Data
(63) Continuation of application No. PCT/US10/21295, filed on Jan. 17, 2010.
(60) Provisional application No. 61/241,868, filed on Sep. 12, 2009.
(60) Provisional application No. 61/150,135, filed on Feb. 5, 2009.

(51) Int. Cl. G01N 33/68

(52) U.S. Cl. 436/88; 436/86

(57) ABSTRACT

Diagnostic devices and methods involve comparison of relative levels of first and second components and/or characteristics of a fluid sample (e.g., saliva), preferably using antibodies arranged to interact with selected components, and colorimetric indicators that are bound or released in proportion to relative concentration or amount of the components or characteristics, as indicative of a health condition such as dehydration state, shock state, stress state, disease state, drug consumption, and drug metabolization. Amylase and IgA may be selected as specific salivary components of interest.

### CROSS-REFERENCE TO RELATED APPLICATIONS

This application is a continuation of International Patent Application No. PCT/US10/21295 filed on Jan. 17, 2010, which claims priority to each of U.S. Provisional Patent Application No. 61/150,135 filed on Feb. 5, 2009 and U.S. Provisional Patent Application No. 61/241,868 filed on Sep. 12, 2009. The disclosures of each of foregoing applications are hereby incorporated by reference herein for all purposes.

### FIELD OF THE INVENTION

The present invention relates to devices and method for diagnosing physiologic conditions using liquid samples.

### BACKGROUND

Shock is the condition whereby the body is not receiving enough oxygen delivery to the tissues. Shock can be due to blood loss, dehydration, or loss of blood pressure. Shock can also be caused by heart problems, insufficient blood volume, allergic reaction, infections, and damage to the nervous system. Shock is life threatening, because if left unchecked, it will cause organ failure and result in death. Unfortunately, shock can worsen and death can occur very rapidly without immediate medical treatment. Therefore, it is imperative that medical professionals be able to quickly diagnose that the patient is suffering from shock

A number of different diagnostics exist for the assessment of hydration state. For example, urine specific gravity is a common standard among certain physicians. For patients that can be monitored over time, total urine output is often used as a metric. Other diagnostics either existing and/or under development look at other potential factors in the urine, blood or saliva.

For many reasons, saliva is an ideal choice for development of a rapid, point-of-care diagnostic measurement for dehydration and/or stress. The sample is easily obtained with minimal invasiveness. No blood must be drawn. In many cases, it is difficult for an individual or health care provider to access urine in a patient (especially for the elderly or infants).

Aside from water content, saliva contains a number of proteins, minerals, salts, peptides, and other small molecules. Two of the most abundant proteins in saliva are IgA and Salivary Amylase. Both of these proteins have been extensively studied in the scientific literature. Amylase is also abundant in the bloodstream (as it is produced by the pancreas) and is a marker for a number of disease states in the blood. A number of diagnostic tools have been developed in the literature for assessing amylase activity in various settings. For example, Salimetrics, LLC (State College, Pa.) sells a benchtop kit for assessing amylase activity in saliva. Molecular Probes Inc. (Eugene, Oreg.) offers a fluorescent kit for assessing amylase activity using fluorescence. These kits utilize the inherent capability of amylase to cleave carbohydrate bonds (salivary amylase converts starches to maltose, one of the first steps in the digestive process). A colored or fluorescent molecule is covalently attached through a carbohydrate bond to a quenching molecule, and the maintenance of such bond renders the colored molecule colorless. When the amylase cleaves the carbohydrate bonds, the colored molecule is released from the quencher, thus adding to the spectral absorbance/emission at a particular wavelength. Various substrates for amylase have been disclosed in the art, such as aromatic substituted glycosides (see U.S. Pat. No. 5,158,872, which is hereby incorporated by reference herein) and 2-chloro-p-nitrophenol linked with maltotriose (Salimetrics α-Amylase Salivary Assay Kit, Salimetrics, LLC, State College, Pa.).

Attempts to extend conventional laboratory techniques to diagnostic applications have been inhibited by various limitations. First, conventional techniques are inherently sensitive to the amount of sample used. For example, amount of signal or indication is generally proportional to the amount of sample (particularly when the amount of sample (e.g., saliva) is small relative to a total test volume), such that if twice as much saliva as desired is applied to a diagnostic device, then typically twice as much signal will be produced. Sampling thus becomes a major obstacle to obtaining accurate test results, since it becomes important to ensure that the same amount of sample (e.g., saliva) is delivered in each test. The potential for false positives and false negatives is great with diagnostic devices used at the point of care.

Second, results of conventional test methods are time dependent, as amylase typically continues to generate additional signal with the passage of time. Thus, if two different tests are allowed to progress for appreciably different amounts of time, the test results can be very different.

Finally, the measurement of a single biomarker in saliva may to provide sufficient signal to noise ratio to overcome variations in the assay technique and/or variations in the biomarker level due to environmental or genetic variations in the sample.

Based on the foregoing, the art continues to seek diagnostic devices and methods adapted to overcome one or more of the foregoing limitations.

### SUMMARY OF THE INVENTION

The present invention relates in various aspects to diagnostic devices and methods involve comparison of relative levels of first and second components and/or characteristics of a fluid sample (e.g., saliva), preferably including use of bound antibodies arranged to interact with selected components, and colorimetric indicators that are bound or released in proportion to relative concentration or amount of the conditions and/or characteristics.

In one aspect, the invention relates to a method of sensing a state of hydration or dehydration of a mammalian subject using saliva provided by or obtained from the mammalian subject, the method comprising: contacting at least a portion of the saliva with at least one first indicator or test region disposed in or on a diagnostic device, and responsively generating a first signal correlative of concentration of at least one analyte in the saliva; contacting at least a portion of the saliva with at least one second indicator or test region disposed in or on the diagnostic device, and responsively generating a second signal correlative of salivary flow of the mammalian subject; and utilizing the first signal and the second signal to assess state of hydration or dehydration of the mammalian subject.

In another aspect, the invention relates to a method of sensing a state of hydration or dehydration of a mammalian subject using saliva provided by or obtained from the mammalian subject, the method comprising: contacting at least a portion of the saliva with at least one first indicator or test region disposed in or on a diagnostic device, and responsively generating a first signal correlative of concentration of IgA in the saliva; contacting at least a portion of the saliva with at least one second indicator or test region disposed in or on a diagnostic device, and responsively generating a second signal correlative of concentration of albumin in the saliva; and utilizing the first signal and the second signal to assess state of hydration or dehydration of the mammalian subject.

In a further aspect, any of the foregoing aspects may be combined with one another or with one features or elements disclosed herein for additional advantage.

Other aspects, features and embodiments of the invention will be more fully apparent from the ensuing disclosure and appended claims.

### BRIEF DESCRIPTION OF THE DRAWINGS

FIGS. 1A-1D are schematic side views of a lateral flow diagnostic device according to one embodiment in four test conditions, the device including antibodies of two types bound on different regions of a solid support, each antibody type being adapted to interact with a different analyte.

FIGS. 2A-2C are top view representations of a lateral flow test strip containing the antibodies represented in FIG. 1 according to three different conditions.

FIG. 3 is a is a schematic view of antibodies of two types bound on different regions of a solid support, each antibody type having an associated label and adapted to interact with a different analyte.

FIG. 4 is a schematic view of the antibodies and solid support of FIG. 3 following displacement by two different analytes of labels previously associated with the bound antibodies.

FIG. 5 is a schematic view representation of antibodies of two types bound on the same region of a test device, each antibody having an associated label and adapted to interact with a different analyte.

FIGS. 6A-6C are top view representations of a lateral flow test strip containing antibodies represented in FIG. 5 under three different conditions.

FIG. 6D is a top view representation of a calibration scale permitting comparison of results obtained from the test regions of FIGS. 6A-6C.

FIGS. 7A-7B are top view representations of a lateral flow test strip or assay device including multiple parallel test regions, showing the device in two different conditions.

FIG. 8 is a bench top titration curve obtained after concentration of Amylase was serially diluted and measured using a commercially available colorometric reader.

FIG. 9 is bench top titration curve for a dilution series of IgA.

FIG. 10 is a plot of weight loss of one experimental subject over four days including one value each for of Days 1-3, and four values for Day 4 at thirty minute intervals before, during, and after a ninety minute exercise period.

FIG. 11 is a plot of salivary Amylase signal of the same experimental subject over four days including one value each for of Days 1-3, and four values for Day 4 at thirty minute intervals before, during, and after a ninety minute exercise period.

FIG. 12 is a plot of salivary IgA signal of the same experimental subject over four days including one value each for of Days 1-3, and four values for Day 4 at thirty minute intervals before, during, and after a ninety minute exercise period.

FIG. 13 is a plot of the ratio of Amylase signal to IgA signal derived from the data of FIGS. 11-12.

FIG. 14 is a plot of the ration of Amylase signal to IgA signal for a different experimental subject that lost 2.4% body weight over a four day experimental study, with one value each for of Days 1-3, and four values for Day 4 at thirty minute intervals before, during, and after a ninety minute exercise period.

### DETAILED DESCRIPTION

It is one object of the present invention to enable a point-of-care diagnostic test to determine the relative level of mammalian (e.g., human or animal) health and/or condition by detecting two different components and/or characteristics of a fluid sample (e.g., saliva) of the mammalian subject and comparing the relative ratio of same. It is another object of the present invention to enable a diagnostic that compares at least two different analytes in saliva as a marker for shock and/or dehydration. It is another object of the present invention to enable a saliva-based diagnostic where sampling differences have minimal effect on the final result. It is another object of the present invention to enable a diagnostic test that is applicable within a given stratum of people and that is independent of environmental issues.

One aspect of the present invention involves the quantitation of at least two different components (e.g., analytes) present in saliva and/or characteristics of saliva. The amounts and/or concentrations of these two analytes or conditions are compared to one another and that ratio determines a health and/or patient condition. Examples of such conditions include, but are not limited to, dehydration state, oral hygiene, oral health, shock state, stress state, disease state, drug consumption, and drug metabolization. Examples of components of saliva that may be considered include minerals, salts, small molecules, proteins, enzymes, peptides, bacteria, and viruses. An example of a condition of saliva that may be considered includes pH. For example, one embodiment of the present invention involves the quantitation of salivary amylase and the quantitation of IgA, which may be compared to one another.

Other analytes in saliva can also be used for these comparisons. For instance, bicarbonate is a major buffering agent in saliva and changes in concentration may affect the pH of the saliva. In another embodiment of the invention, the concentration of one analyte is compared to the concentration of bicarbonate by measuring the pH and comparing to the concentration or activity of another analyte. Another salivary digestive enzyme is lingual lipase. The concentration of this protein could also be used in the comparison. Other salivary enzymes include, but are not limited to, mucins and epidermal growth factors. These could also be used for diagnostic comparison. Total protein count in the saliva could also be used as one of the markers, as could total plate count (e.g., bacteria concentration). Analytes that are not generated by salivary glands but are instead transferred into the oral cavity from serum, such as serum albumin or serum circulating drugs, can also be used.

In one embodiment of the present invention, the concentration of two different analytes in saliva is compared and the result of this comparison indicates a relative level of hydration/dehydration. However, other health factors can be determined using this comparison. For example, certain medications increase or decrease certain protein and analyte production in saliva. Thus, a test according to the present invention could be used to indicate adverse affects of medication. Additionally, it could be used as a marker to determine whether medicine is being administered as prescribed and/or being metabolized by the patient. For example, while a patient is on certain medication a baseline comparison of two analytes may be determined. In the future, if the medicine is not administered as proscribed, then the relative concentration of the two analytes may change from the baseline. Additionally, alcohol has been shown to effect saliva composition (see Brand, H. S. et al., Int. J. Dental Hygiene, 4 (2006) pp 160-161). Thus, a test according to the present invention could be used to detect alcohol consumption. Some studies have also indicated that the level of analytes in saliva could be a marker for oral hygiene. Detection or characterization of additional health conditions or health-related factors is envisaged, as will be recognized by one skilled in the art.

Many different assay formats can be used in device and methods according to the present invention. In certain embodiments, the relative concentration of the two analytes will generate a color change on a test strip or in solution. The intensity of the color can be the indicator of concentration level. In one embodiment, the two analytes generate the same color and the intensity of each color is the indicator. In this embodiment, the color generated by each analyte would be physically separated from each other. In other embodiments, each analyte will generate a different color. Similarity or difference between colors of different test regions may be correlative of a selected health condition. In a preferred embodiment, the color changes are detected by visual comparison of a user. In other embodiment, a reader (such as a UV visible spectrophotometer, absorption measurement device, light scattering device, fluorescence reader, etc.) may be used to quantitate the presence (e.g., concentration and/or amount) of analytes. Such a reader can be a bench top reader or a hand held device. If a reader is used, the reading device may also store the results over time and/or be in communication with a computer or other storage media.

Certain embodiments involve use of a lateral flow diagnostic test strip. A user may apply saliva to an active area of the device. Alternatively, a cassette can be utilized, wherein a user puts an area of the device into the user' s mouth to collect saliva on a portion of the lateral flow assay device (such portion may be devoid of any reagents). Capillary action may then wick the collected saliva into a different portion of the device containing immobilized reagents. A lateral flow membrane may thus be employed as a sample transport element. Other sample transport elements may be used, including pressure-based fluid movement (whether by manual manipulation of a device, or motivated by a machine element—such as one arranged to provide peristaltic pumping action). In one embodiment, a test strip or assay device may be supported in a substantially vertical orientation by a holding (not shown) arranged to permit gravitational forces to transport sample within the device.

In another embodiment, an assay is normalized and sensitized to a given patient population and/or stratification. For example, it may be determined that certain patient populations have naturally higher concentrations of a certain analyte than other groups of patients. Additionally, selected patient groups may exhibit lower levels of a different analyte. Thus, it may be desirable to consider different ratios of two different analytes than listed above in the Amylase/IgA example. Different assays may be developed for each of these populations wherein a “normal” indication would appear when the ratio of the selected analytes is at or near the normal ratio for the selected population. Thus, for example, one assay may be used for children and a different assay may be used for adults.

Populations may be stratified by a number of factors, including but not limited to: age, sex, pregnancy, ethnicity, weight, height, etc. Temperature of a mammalian subject at the time a sample is obtained may also be used to define an applicable patient population.

In another preferred embodiment of the invention, an assay is normalized to certain environmental factors. For example, it may be determined that during high levels of exercise, the ratio of two selected analytes differs in a hydrated state than when the individual is at rest. For example, at rest the ratio of two selected analytes may be 2:1, whereas during exercise (but while the individual is still in a hydrated state), the ratio becomes 5:2. Thus, an assay specific for use during exercise may be constructed, wherein a ratio of 5:2 appears normal, and departure from that normal ratio identifies a selected health condition such as (but not limited to) dehydration. Such device may be specifically designed for use by individuals while exercising.

Other environmental normalization can be envisioned. For instance, individuals with a certain disease state may have different “normal” ratios of salivary analytes than healthy individuals. Assays can be developed for individuals exhibiting different disease states (such as cancer, common cold, stomach flu, etc.).

One potential application of the present invention is for parents and/or health care providers to check the hydration level of sick children and infants. As an example, children having the flow may vomit, and health care providers are often concerned that the child will become dehydrated due to such vomiting. For this end use, the ratio of two analytes may be affected by remaining vomit in the child's saliva and/or pH changes in the saliva due to the vomiting. Thus, an assay for this application may look at normal analyte levels during a hydrated state for a child with the flu who had recently vomited. Levels of the analytes during an unhydrated state after recent vomiting would also be determined and that level would be the marker for dehydration.

Environmental factors that can be stratified include, but are not limited to, health, medication being taken, diet, liquid consumption, temperature, caffeine intake, alcohol intake, time of day, etc.

In another embodiment, an assay can be normalized to a specific person. In this embodiment, an assay may be used to test the hydration level of an individual over time. For example, elderly people may be checked on a daily basis. Athletes may check themselves at multiple times during exercise. In this embodiment, individuals may check themselves one or more times when they are in a hydrated state. The ratio of two analytes at that time would be noted or recorded. Then, when an individual is checked in the future, such individual would compare the result to the individual's own personal baseline level.

In one embodiment, a series of tests may be constructed either on the same solid support or on different solid supports. Different text regions on the same device (or different tests) may be normalized to given patient stratifications. For instance, a single test strip could have multiple (e.g., three, four, five, ten, or any desired number) different test regions for different populations stratified by things such as age, weight, ethnicity, general health, medication, etc.

Such a test can be normalized to a given population as follows. Consider an adult male population where the healthy baseline ratio of salivary amylase to IgA is 2:1 and the unhealthy level is above 2.5:1. An immunoassay format may be used to make the analyte comparison. In one embodiment, the immunoassay may be a simple competition assay. In this embodiment, an antibody against amylase may be incubated with a 50/50 ratio of a colored substrate and uncolored substrate that both also bind to the antibody (but with a weaker binding constant). This antibody substrate complex is immobilized (either covalently or non-covalently) on a solid support (such as a filter paper) in a specific location (e.g., a first test location). A second antibody against IgA is then incubated with the colored substrate only (the same color as the substrate above) and also bound to the solid support but in a different location (e.g., a second test location, which may be disposed close to the first test location to facilitate visual comparison).

This assay may be normalized to the 2:1 ratio listed above. When 100 units (example for clarity only, not necessarily representing a specific amount) of amylase come in contact with the immobilized antibodies, they will release 50 colored substrates and 50 uncolored substrates. When 50 units of IgA come in contact with the region, they release 50 units of colored substrate. Thus, when the saliva contains a 2:1 ratio of amylase to IgA, the first and second test regions will appear to be the identical color, or will otherwise give a similar reading with a reader. Thus, in this embodiment, the relative intensity of the two test regions is determined, and represents the relative concentration of the two analytes. Such normalization may also be performed using a smaller amount of labels for the analyte that is present at a higher concentration.

In another embodiment, two different colors may be used for each of two analytes. For instance, consider analyte A and analyte B in saliva that are present in a 1:1 ratio for a patient in a healthy state. As in the preceding embodiment, antibodies against these two analytes will be utilized. However, in this embodiment, an antibody against analyte A may be incubated with a colored substrate (such as one that is blue in color) and analyte B may be incubated with a different colored substrate (such as one that is yellow in color).

In this embodiment, antibodies may be immobilized in the same location on a solid support or present in another medium, such as a tube or other apparatus. Such embodiment involves a first analyte test or detection region that is at least partially coextensive with a second analyte test or detection region. When a sample is applied to the support, the analytes will displace the colored substrates from the antibodies and liberate them into solution, thus producing color. The higher the concentration of the analyte, the more of the respective color will be generated. The resulting color will correspond to the ratio of analyte A to analyte B. When the colored substrates are blue and green, a ratio of 1:1 between analytes A and B may result in production of a true green color in the test region, resulting from combination of equal parts of blue and yellow. If the color of the test region is more blue than green, then that indicates higher concentration of analyte B. Conversely, if the color of the test region is a yellowish green color, such condition would indicate a higher concentration of analyte A.

In another embodiment, an assay may allow an individual or health care provider to normalize a given analyte ratio for an individual. Although this embodiment refers to monitoring levels of IgA and amylase, other analytes may be used.

This embodiment involves use of an immunoassay format substantially similar to that described immediately above, wherein blue and yellow substrates are liberated by amylase and IgA, respectively. When saliva containing amylase and IgA comes in contact with a test region having immobilized antibodies arranged to release blue and yellow colored substrates, respectively, a greenish color may be produced. The relative amount of yellow and blue substrate released will be indicative of the ratio of IgA to amylase. The relative presence of amylase to IgA provides indication of health issues and/or dehydration. Extending this embodiment to a particular assay device, a lateral flow test strip can be housed in a plastic housing. On a plastic carrier next to the test region where the antibodies are immobilized, a color scale strip or other reference scale (e.g., reference color scale) with gradients from pure yellow to pure blue (e.g., including shades of green between), preferably including having associated numbers (e.g., 1-10) or other indicators correlative of at least one selected condition. When the sample indicates a normal hydrated state, the individual or health care provider notes the number corresponding to the color produced from the saliva (sample). Such action may be repeated and averaged to provide a baseline or trend. Thereafter, new test results (e.g. obtained with other assay devices) may be compared to the original baseline number established for the same individual or patient. For example, a test device or method may be provided wherein a selected health condition such as dehydration is correlated to an increase of two units on such an assay. A healthy male individual may establish a hydrated baseline of four units on the above scale. Thus, if the same individual is tested in the future and such test generates a result of six units, then such result provides indication that the individual has a hydration problem (or other health related condition). Using the same assay format, a pregnant woman may generate a baseline level of six units. Subsequent testing providing a result of eight units or more would provide indication of a health or hydration issue.

In another embodiment, a different assay format may be used. Two sample characteristics to be tested include amylase concentration and pH (which is a marker for bi-carbonate). Bi-carbonate is the major buffering source in saliva. The pH of saliva generally goes down during states of low salivary flow, such as when an individual is dehydrated. In this embodiment, a cleavage assay may be used for amylase and a pH test region may be provided for bi-carbonate. A chromagenic substrate (such as 2-chloro-p-nitrophenol linked to maltotriose) is cleaved by the amylase to release a colored substrate (in this case at 405 nM). Assay kits of this type that monitor amylase activity are commercially available. The substrate may be immobilized on a test region of a diagnostic device (e.g., a test strip). A pH sensitive assay is also immobilized in another region of the strip. A pH assay is chosen that also produces signal at 405 nM. As before, the relative intensity of the signal at 405 nM may be normalized for pH and amylase concentration so that the two different test regions appear to be the same color during a normal hydrated state. The relative color of the two regions would be different when the individual from whom the sample was obtained experiences health or hydration problems.

The foregoing and other concepts are further described in connection with the appended figures.

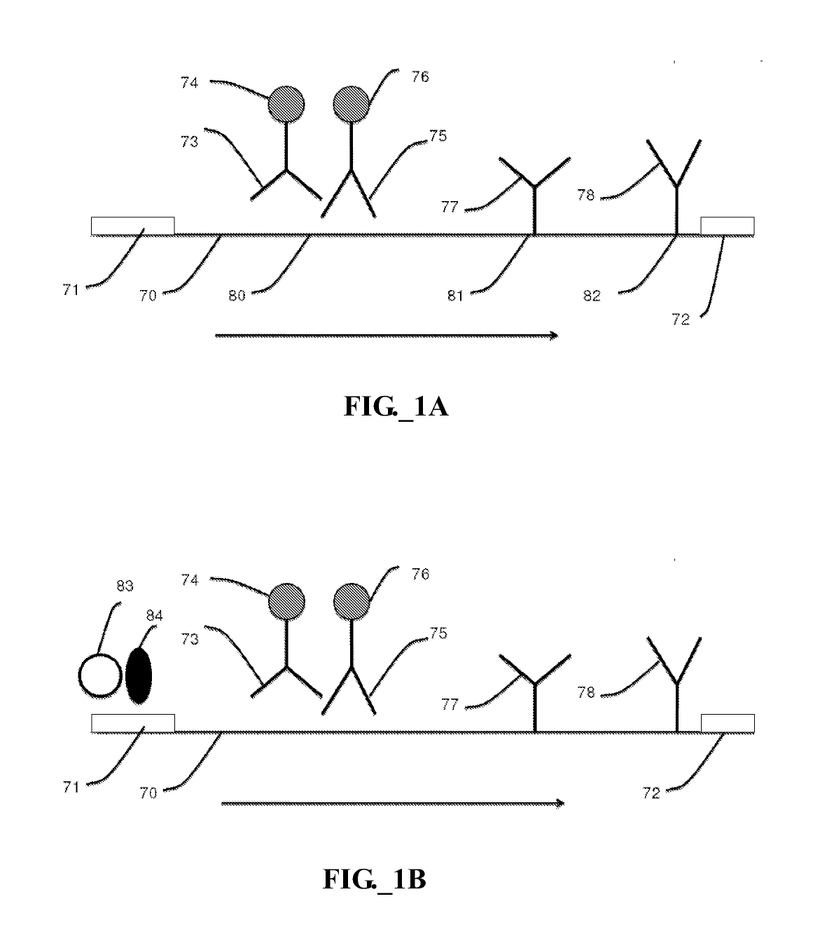

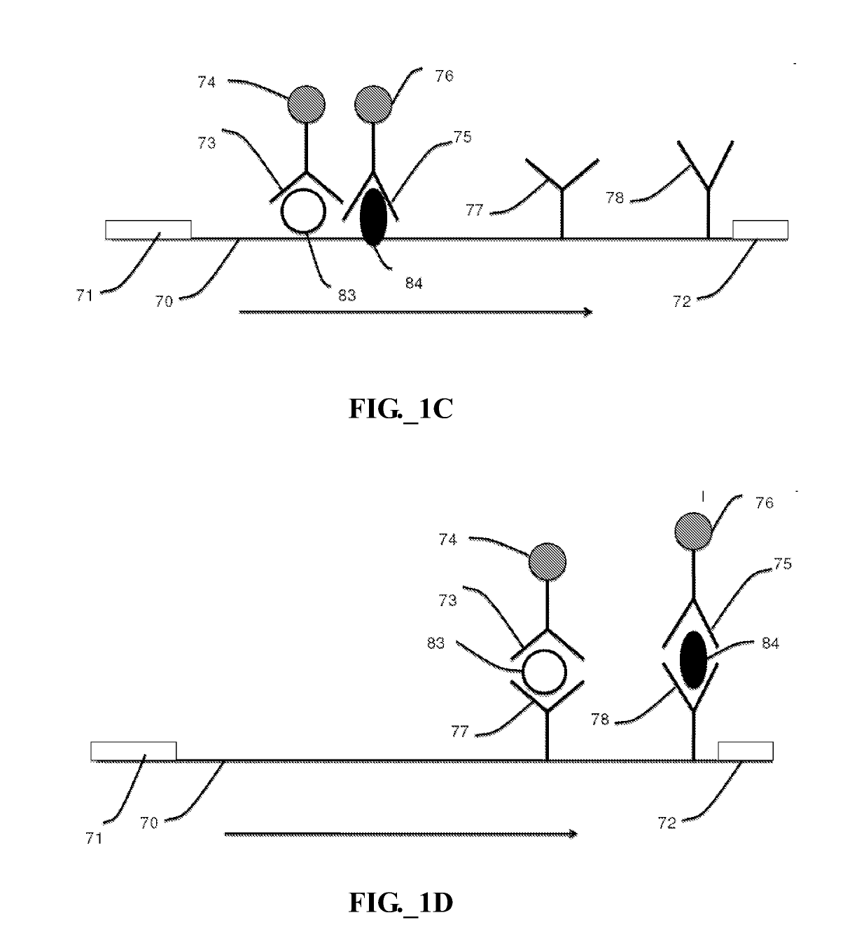

FIGS. 1A-1D depict a lateral flow diagnostic device comprising a sandwich assay according to one embodiment of the present invention, in four different test conditions. A lateral flow membrane material 70 such as nitrocellulose provides the basis of the device where a sample will flow. Sample may be applied to a sample receiving or admission region 71, which may include an absorbent material that is preferably devoid of assay reagents. A labeling region 80 of the device may be preincubated with labeling antibodies, such as antibodies 73, 75. These antibodies 73, 75 may be simply dispensed onto the labeling region 80 and allowed to dry during the manufacture; the antibodies are preferably not bound to the membrane 70. A first antibody 73 that binds selectively to amylase in labeled with a first colored reagent 74. This first colored reagent 74 may include, for example, gold nanoparticles, colored latex beads, colored chemical moieties, etc. A second antibody 75 selectively binds IgA and is labeled with a colored reagent 76 that may be the same color as the first colored reagent 74. In a first test region 81, additional antibodies 77 that selectively interact with amylase are attached so that they are bound to the membrane 70. These bound antibodies 77 may be the same type as the first antibody 73 used for the labeling, or the bound antibodies 77 may differ in type from the first antibody 77. In a second test region 82, antibodies 78 that selectively interact with IgA are bound. These bound antibodies 78 may be the same type or different than the second antibody 75 used for labeling. A sample terminus region 72 may be provided downstream of the bound antibodies 77, 78 relative to the direction of travel of sample along the substrate 70. The direction of travel of sample through or on the device is depicted by the (rightward) arrow provided below the substrate 70. Different reagents (such as surfactants) may be applied to portions of the device in order to enhance fluid flow, block non-specific protein binding, enhance stability, and provide other beneficial effects, as will be recognized by one skilled in the art.

FIGS. 1B-1D schematically illustrate molecular interactions as the device is used. In FIG. 1B, sample (e.g., saliva) is applied to the sample receiving or admission region 71. In this example, the sample is saliva that contains both amylase 83 and IgA 84. The membrane material 70 is porous and is designed to allow the sample to be drawn through capillary forces down the device toward the sample terminus region 72. In FIG. 1C, the schematic shows what happens as the sample passes the labeling region 80. In this example, the sample contains both analytes and they bind to corresponding antibodies 73, 75. As the sample progresses along the membrane 70, these analyte/antibody conjugates are carried with it. When the conjugates come into contact with the corresponding bound antibodies 77, 78 in test regions 81, 82, the conjugates bind to the bound antibodies 77, 78 and the colored labels 74, 76 are thus spatially bound to the test regions 81, 82. This assay format is typically referred to as a “sandwich assay.” If one of the analytes 83, 84 was not present, the corresponding labeled antibody would continue to progress down the membrane 70 until it encounters the sample terminus (e.g., adsorbent) region 72, which is outside the test or viewing section of the diagnostic device.

In one embodiment, saliva provided by or obtained from a mammalian subject may be at least partially dehydrated, and such dehydrated or partially dehydrated saliva may be at least partially rehydrated prior to contacting same with a sample receiving or admission region of an assay device.

In FIGS. 1A-1D, only one of each molecule is shown for purposes of illustration; however, in an actual device, many molecules of corresponding types would be located at each step of the progression to produce sufficient signal to be observable by a reader or human eye.

The foregoing illustrative example involves comparison of amylase to IgA in a 1:1 ratio. A darker resulting color at each test region 81, 82 is correlative of amount or concentration of the selected analyte (e.g., amylase at first test region 81, and IgA at second test region 82). The device can be calibrated with standards and a color chart to enable quantitation of the amounts of the selected analytes in the sample. Additionally, as noted herein, it may be desirable to normalize the device against known healthy ratios of analytes in a given patient population. For example, it may be represented in literature that normal ratios of amylase to IgA in healthy adult males is 2:1. In such example, 50% of the antibodies against amylase 73 at the labeling region 80 may be provided without any colored conjugate. Thus, if twice as much amylase was bound at the first test region 81 compared to IgA bound at the second region 82, the test regions 81, 82 would appear identical in terms of the color concentration, since half the bound amylase would be unlabeled. Alternatively, all of the antibodies 73, 75 may be labeled, but a smaller number of antibodies for one analyte may be applied to a test region until the device is normalized appropriately.

For a variety of reasons, it may be necessary to have different (e.g., larger) percentages of the antibodies be devoid of labeled conjugates. For example, it may be appropriate to have 10% of the IgA antibodies labeled and 5% of the amylase antibodies labeled.

Many different methods of applying sample to the sampling receiving or admission region 71 may be employed. For example, an end of a diagnostic device may be dipped in a sample to promote contact. In such example, an individual deposits saliva into a cup or other container, and the end of a test strip may be dipped into the saliva. It may be advantageous to mix some other fluid, such as water or buffer, into the saliva prior to introduction of same into a diagnostic device. Likewise, it may be desirable for an individual to administer water or other liquid in their mouth and swirl it around prior to depositing saliva into a container for sampling.

An end of a diagnostic device, such as the sample receiving or admission region 71, may alternatively be placed into the mouth of an individual to receive saliva. Alternatively, a swabbing device could be used to obtain saliva. This swab could then be dipped into a container containing extraction liquid, such as water or buffer, so that analytes of interest are transferred into this extraction liquid. The sampling region 71 could then be dipped in the extraction fluid.

In one embodiment, a diagnostic device such as shown in FIGS. 1A-1D may be fitted into an associated (e.g., plastic) cartridge. Such cartridge may expose the sample receiving or admission region 71 to an outside environment and could enclose the remainder of the device to prevent tampering with or damage to the labeling region 80 and the test regions 81, 82. The test regions 81, 82 could be viewed through an optically transmissive portion (e.g., a transmissive window or hole) of the cartridge.

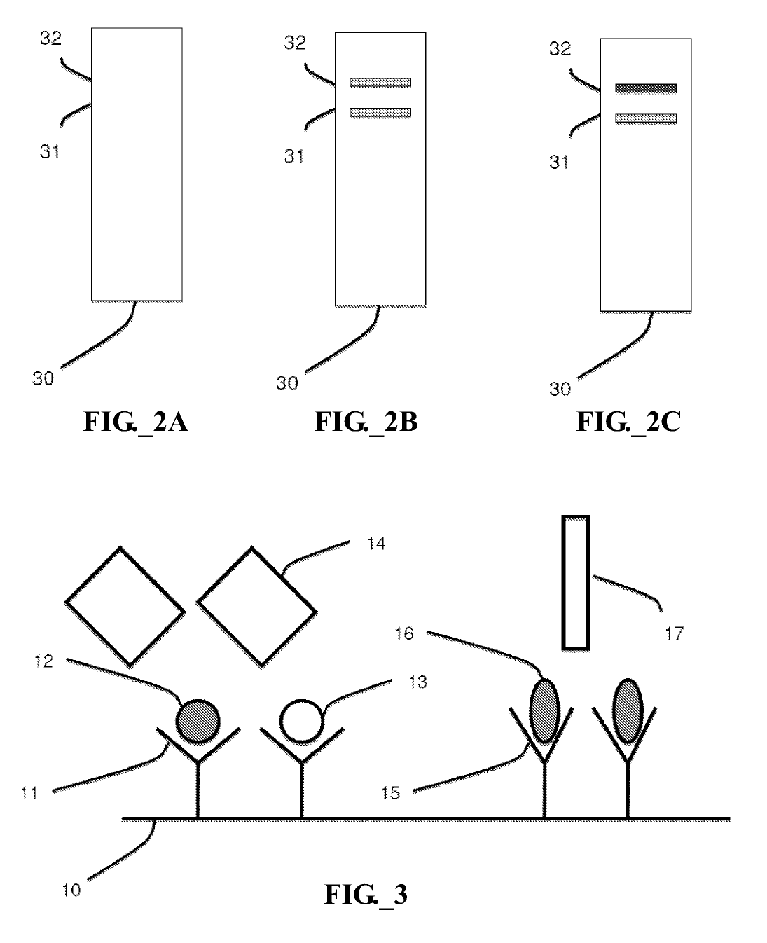

FIGS. 2A-2C provide top view representations of a lateral flow assay device similar to the device illustrated schematically in connection with FIGS. 1A-1D. In such device, antibodies may be placed in first and second test regions 31, 32 on a solid support 30. In this example, antibodies against a more abundant first analyte are bound in a first test region 32 (i.e., closer to one edge of the support 30), and the antibodies against a less abundant second analyte are placed in a second test region 31 (i.e., closer to the center of the support 30). Only half of any unbound antibodies (i.e., in a labeling region of the device) for the first analyte have an associated colored label. FIG. 2A shows the assay device prior to the application of a sample.

FIG. 2B shows the assay device following addition of a sample containing a first analyte at twice the concentration of the second analyte. In FIG. 2B, both test regions 31, 32 will be developed in the assay and will be the same overall color, despite the higher concentration of the first analyte, since only half of the conjugates freed by first antibodies upon addition of the first analyte contain a colored label.

FIG. 2C shows the assay device following addition of a sample having an even higher concentration of the first analyte 14. In this example, the test region 32 for the first analyte is much darker than the second test region 31 for the second analyte.

In certain embodiments, the intensity difference between the two test regions of the lines may be sufficiently large for the human eye to determine the outcome of the test. In another embodiment, an optical reading element or scanning device may be used to generate signals indicative of intensity difference of for the test regions, and such signals may be compared to determine the outcome of such comparison.

The foregoing simplified description of a lateral flow assay is provided to illustrate functional principles of such assay. As will be appreciated by one skilled in the art, other components and features of lateral flow assay devices and methods are well-known in the art, and incorporation of such known components and features into devices and methods disclosed herein is specifically contemplated.

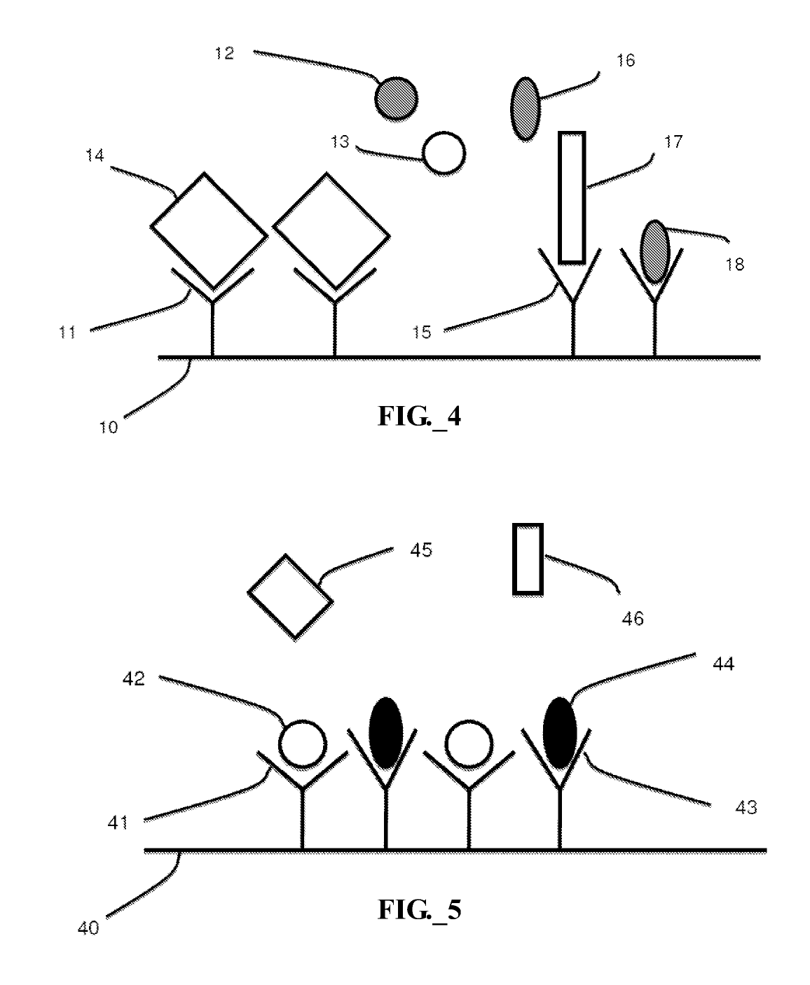

FIGS. 3-4 schematically illustrate a portion of a lateral flow test strip (assay device) and test method according to another assay format called a displacement assay. For brevity, various portions of the device similar to FIG. 1 have been omitted and only the test regions are shown. FIG. 3 is a schematic view of a portion of a support surface 10 of a lateral flow assay device. The support surface 10 has bound thereto antibodies 11, 15 against (i.e., adapted to interact with) two different analytes 14, 17, with each antibody type being bound to different physical regions of a lateral flow test strip including the support surface 10. Although FIG. 3 depicts analytes 14, 17 for illustrative purposes, it is to be assumed for purposes of FIG. 3 that such analytes 14, 17 are not yet available for interaction with antibodies 11, 15. A first antibody 11 is adapted to interact with a first analyte 14, and a second antibody 15 is adapted to interact with a second analyte 17. Prior to placing the antibodies 11, 15 onto the surface 10, the first antibody 11 is preferably incubated with different conjugates 12, 13, and the second antibody 15 is preferably incubated with another conjugate 16, wherein the conjugates 12, 13, 16 are selected to bind to the respective antibodies 11, 15 but at a weaker equilibrium constant than the analytes 14, 17.

In the case shown in FIG. 3, for illustrative purposes the first analyte 14 is present in a sample (i.e., intended for introduction to the assay device along the surface 10) at twice the average concentration of the second analyte 17. To accommodate such difference in analyte concentration, the portion of the test strip or assay device that contains the first antibody 11 (i.e., adapted to interact with the first analyte 14) may be has been bound with two different conjugates 12, 13. One conjugate 12 has a colored substrate attached thereto. The other conjugate 13 does not have an associated colored label. For the test portion of the assay device having the antibody 15 (i.e., adapted to interact with the lower concentration analyte 17), each conjugate 16 has an attached label.

In this embodiment, the colored labels used with conjugates 12, 16 may be the exact same label. FIG. 4 is a schematic view showing the antibodies 11, 15 and solid support 10 of FIG. 3, following presentation in the lateral flow assay of a sample containing both analytes 14, 17, thus making the analytes 14, 17 available to interact with the antibodies 11, 14, respectively. In this case, twice as much of the first analyte 14 is present as the second analyte 17. The analytes 14, 17 displace the colored conjugates 12, 16 and non colored conjugate 13 from the antibodies 11, 15. The displaced color generates a signal correlative of amount or concentration of the analytes 14, 17.

In another embodiment of the present invention, antibodies against different analytes may be bound to a test strip (or support surface) in or along the same region of an assay device. Referring to FIG. 5, the active region 40 of an assay device 40 may be used to test two analytes 45, 46 that are present in the same concentration for a healthy subject (individual). A first antibody 41 is adapted to interact with a first analyte 45, and a second antibody 43 is adapted to interact with a second analyte 46. The antibodies 41, 43 may be premixed and the resulting mixture may be bound to an active or test region of an assay device. Colored conjugates 42, 44 may be associated with the antibodies 41, 43, and subject to competition with the first and second analytes 45, 46 relative to binding with the antibodies 41, 43. Upon presentation of the analytes 45, 46, the conjugated labels 42, 44 may be freed from the antibodies 41, 43 surface and thereby produce a signal. In one embodiment, the two conjugates 42, 44 may be labeled with colored substrates embodying two different colors.

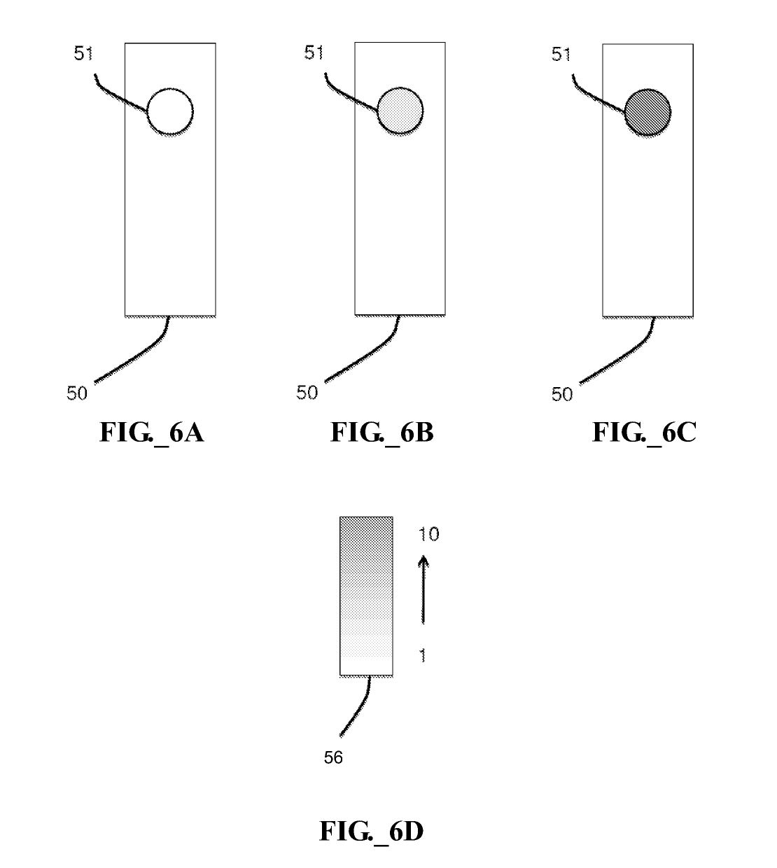

FIGS. 6A-6D provide top view representations of at least the active region of an assay device, including the antibodies and molecules described in connection with FIG. 5. In this embodiment, the antibody mixture containing both antibodies 41, 43 may be bound to the solid support 50 in a circular test region 51. FIG. 6A shows the assay device prior to addition of sample, wherein no color (signal) is generated in the test region 51. FIG. 6B shows the assay device following addition of a sample including two analytes, wherein the ratio of the two analytes is approximately the same, producing a light color in the test region 51. FIG. 6C shows the assay device following addition of a sample including two analytes, wherein the second analyte is present at a higher concentration, thus liberating more color-labeled conjugate. The active region 51 of the assay device has a more prominent color corresponding to the liberated and color-labeled conjugate.

FIG. 6D shows a pre-calibrated gradient 56 (e.g., reference color scale) enabling a user to compare the color in the active region 51 of the assay device to the gradient 56 any determine the outcome of the assay. The gradient 56 may also include numbers, letters, symbols, or other calibrating indicia, such as the numerical scale shown adjacent to the gradient 56.

FIGS. 6A-6C could also represent the viewing portion of a lateral flow device sandwich assay similar to the assay illustrated in FIGS. 1A-1D. Referring back to FIGS. 1A-1D, and applying concepts disclosed therein to the embodiment with a single test region shown in FIGS. 6A-6D, the labeling antibodies 73, 75 would not be conjugated with the same colored label; rather, they may be conjugated with two different colored labels. For example, amylase antibodies could be conjugated with yellow colored latex beads and IgA antibodies could be colored with blue colored latex beads. In this example, the binding (test) regions 81, 82 of FIGS. 1A-1D may be consolidated into the single test region 51 of the diagnostic device of FIGS. 6A-6C, with both sets of capture antibodies 77, 78 (shown in FIGS. 1A-1D) being immobilized in the single test region 51 of FIGS. 6A-6C. The operation of the lateral flow assay would then proceed similarly as described in connection with FIGS. 1A-1C, but instead of measuring the intensity of two different lines of the same color, the test administrator would assess the total color output of the device, such as a gradient of green in this example, to determine the relative concentrations of amylase and IgA. That is, a first color correlative of concentration of a first analyte is interspersed or mixed with a second color correlative of concentration of a second analyte, with the combination yielding a third color. The third color may be compared to a reference scale, such as the reference color scale 56 provided in FIG. 6D, to provide indication of relative concentrations of the first and second analytes.

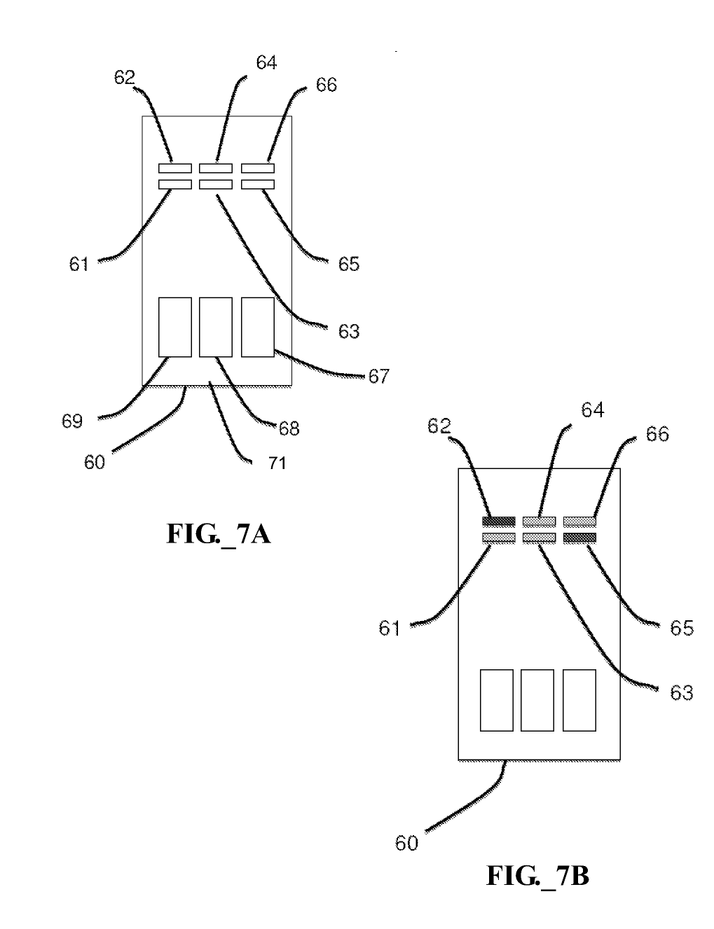

FIG. 7A-7B provide top view representations of a lateral flow assay device as mentioned in connection with FIGS. 1A-1D, but including six different binding (test) regions 61-66 on a solid support 70, with three groups of two test regions each. In this example, antibodies arranged to interact with a first analyte (e.g., amylase) are placed in three test or detection regions 61, 63, 65 on a solid support 60. Antibodies arranged to interact with a second analyte (e.g., IgA) are placed in three further test or detection regions 62, 64, 66. A first pair of test (or detection) regions 61, 62 is arranged to receive a first portion of a sample, a second pair of test (or detection) regions 63, 64 is arranged to receive a second portion of a sample, and a third pair of test (or detection) regions 65, 66 is arranged to receive a third portion of a sample. Each pair of test or detection regions 61-62, 63-64, 65-66 has an associated upstream labeling region 69, 68, 67, respectively. In this example, each detection region pair and associated labeling region 61-62-69, 63-64-68, 65-66-67 may be calibrated for different patient populations by changing the ratio of labeled and unlabeled antibody tags located in the labeling regions 69, 68, 67. For example, it might be found that healthy hydrated infants exhibit a ratio of selected analytes (e.g., amylase to IgA) of 1:1, healthy hydrated adults exhibit a ratio of 2:1 of the same analytes, and health hydrated elderly adults exhibit a ratio of 3:1 of the same analytes. In this example, the first pair of detection regions 61-62 may be calibrated for infants, and an associated upstream first labeling region 69 would contain labeled antibodies for the selected analytes (e.g., amylase and IgA), with the antibodies being labeled with equal proportions of colored conjugates. Note that not every antibody needs to be labeled; the fraction of labeled antibodies may be selected to provide a desired dynamic range. For purposes of the present example, however, the first labeling region 69 may contain first and second antibodies having the same ratio of labeled to unlabeled. A second labeling region 69 may include antibodies calibrated for adults. Thus, in this region 69, antibodies for one analyte (e.g., IgA) may be labeled twice as often as antibodies for a second analyte (e.g., amylase). Finally, in a third labeling region 67, antibodies for one analyte (e.g., IgA) may be labeled three times more frequently than antibodies for another analyte (e.g., amylase), to calibrate this labeling region 67 for an elderly population. As mentioned previously, differing amounts of labeled antibodies in the labeling regions 67-69 could also be employed.

FIG. 6A shows the assay device prior to the application of a sample. Sample would be placed on the lateral flow assay at least one sample receiving region 71. The at least one sample receiving region may include a single region arranged to supply portions of a sample to each labeling region 67, 68, 69, or may include multiple discrete sample receiving regions each separately arranged to supply portions of a sample (or different samples) to the different labeling regions 67, 68, 69.

FIG. 6B shows the same assay device as depicted in FIG. 6A, following addition of a sample containing adult saliva having a 2:1 ratio of two selected analytes (e.g., amylase and IgA). As depicted in FIG. 6B, the middle pair of detection regions 63-64 appear to be the same color. Referring to the leftmost pair of detection regions 61-62, the upper detection region 62 appears to have much more IgA than amylase (which it does) but this pair of detection regions 61-62 is pre-calibrated for a sample received from an infant, and such pair of detection regions 61-62 may be ignored when a sample obtained from an adult (i.e., non-elderly adult) is applied to the device. Likewise, referring to the rightmost pair of detection regions 65-66, the lower detection region 65 appears to indicate a higher presence of IgA than amylase. Since this pair of detection regions 65-66 is calibrated for a sample obtained from an elderly individual, however, such regions 65-66 may be ignored when a sample obtained from a non-elderly adult is applied to the assay device.

In one embodiment, a diagnostic or assay device includes a plurality of regions having differing amounts or concentrations of at least one of the first indicator and the second indicator. At least one region is selected be employed in performing a colorimetric comparison based on at least one factor selected from the group consisting of: time of day the saliva was provided by or obtained from the mammalian subject, or status of age, sex, ethnicity, pregnancy, weight, height, or temperature of the mammalian subject

In another embodiment, a series of carriers may be developed to cover the test or detection regions not applicable to a given population. For example, a single lateral flow test strip 60 may include parallel labeling regions and detection regions calibrated for different populations as shown in FIGS. 7A-7B, and such test strip may be inserted into any of three different carriers (e.g., covering devices) that each include different windows or openings to reveal detection regions appropriate for a given target population, while covering detection regions not applicable to the selected target population. In this manner, a single test strip may be manufactured economically in large volume, and inserted into different tailored carriers.

As demonstrated in connection with FIGS. 7A-7B, an assay device may be provided with multiple different detection regions calibrated for different user populations. It is to be appreciated that an assay device may also be provided with multiple different detection regions calibrated for different health conditions for the same user populations. That is, an assay device may include test regions enabling performance of many assays in parallel from a single sample, with each assay arranged to indicate a different health condition.

In another embodiment of the present invention, two different biomarkers in saliva that both increase in concentration during dehydration or other unhealthy state are measured. Alternatively, two markers that both decrease in concentration under like conditions could also be used. In one embodiment, the assays are set up so that one of the indicators is inverted. That is, one assay will produce a larger signal when the marker concentration increases and the other assay will produce a smaller signal as the marker concentration increases.

For example, a lateral flow assay may be set up in a binding format for a specific protein such as salivary Amylase. A signal on a test strip in this format will increase in intensity at higher Amylase concentrations since more labels will be bound to the test site. A second test strip can be set up in a competitive binding mode where a protein blocks binding of the label. Thus, the test area is less intense in color when higher concentrations of the protein is present.

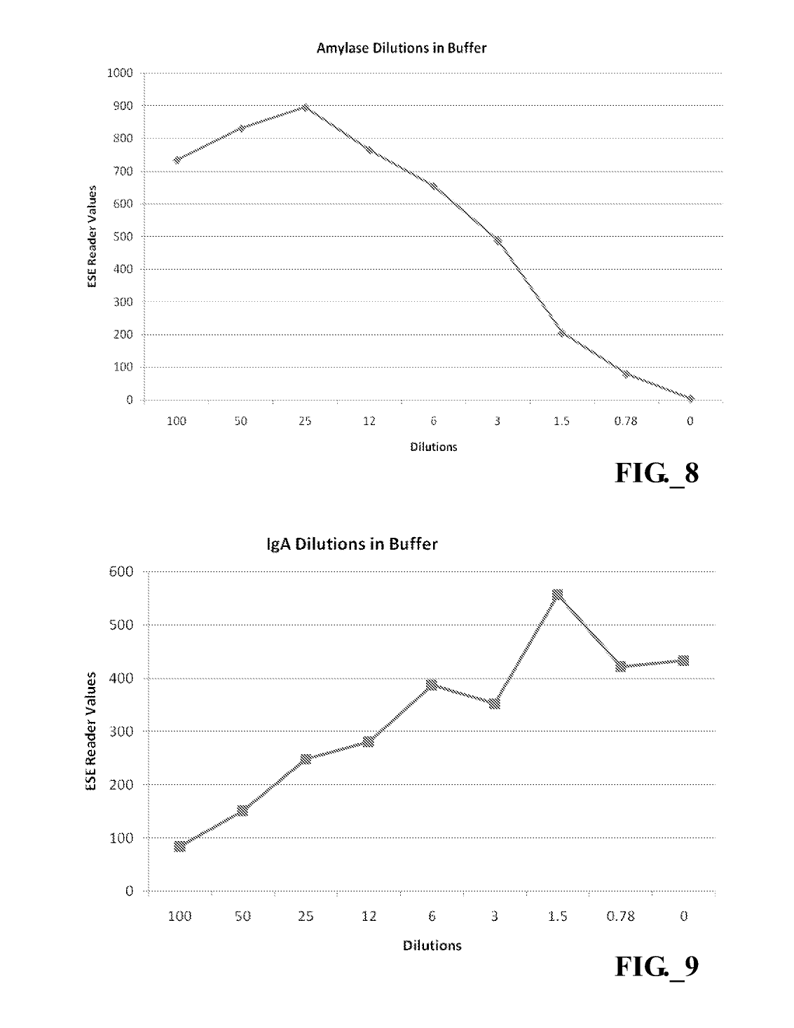

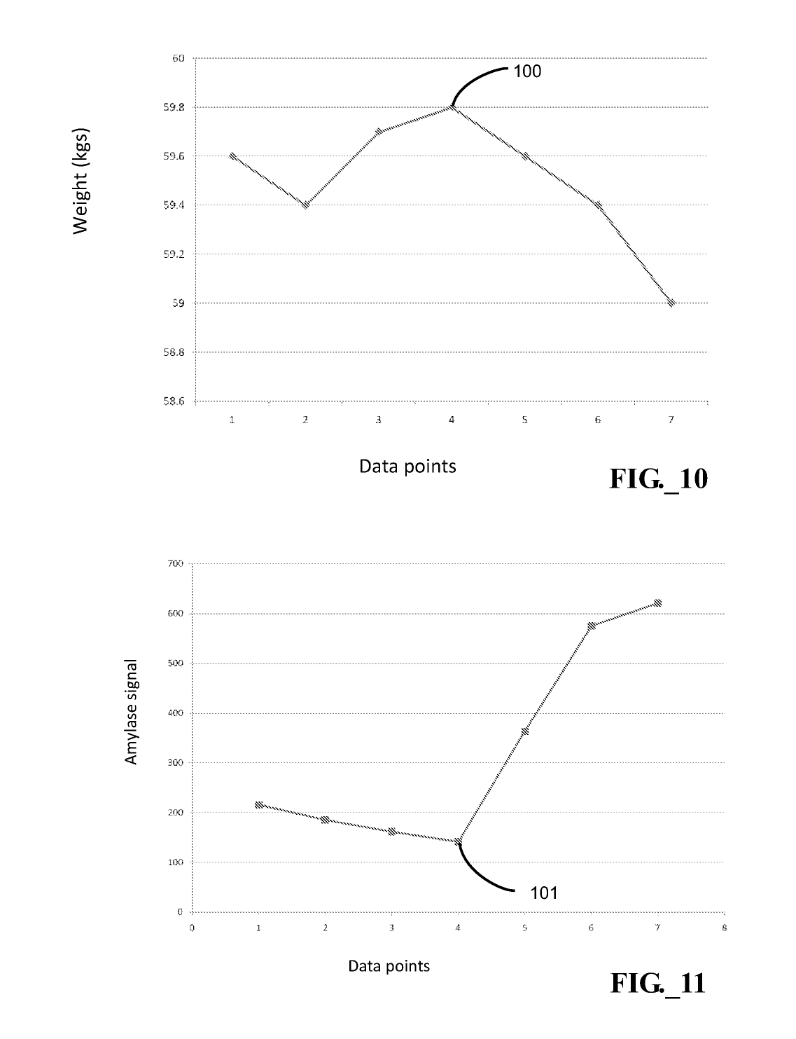

Two separate test strips were constructed as described above. (Note that in another embodiment, separate tests may be integrated onto the same strip in different physical locations). A first test strip was constructed in a binding mode for salivary Amylase. A second test strip was constructed to measure IgA in a competitive mode. FIG. 8 shows a bench top titration curve obtained after concentration of Amylase was serially diluted and measured using a commercially available colorometric reader (ESE Quant from ESE GmbH, Germany). This device was set up using a reddish colored latex bead as the label. As noted from FIG. 8, Amylase signal decreases as the concentration is lowered. FIG. 9 shows a bench top titration curve for a dilution series of IgA, again using the ESE Quant reader to quantitate the results. As indicated in FIG. 9, the IgA signal increases as its concentration is lowered. The Amylase and IgA test strips have been set up in opposite formats.

Healthy human adults were recruited for a four day experimental study utilizing the above-referenced test strips for salivary Amylase and IgA. For the first three days (Days 1 to 3), the test subjects came into the laboratory and a number of physiological conditions were monitored. On each day, each subject's weight, heart rate, blood pressure, and urine specific gravity were recorded. Additionally, each subject's saliva was diluted 60:1 with PBS buffer and tested using the lateral flow assays described above. On the fourth day (Day 4), the subjects were again tested as before. Then, each subject exercised on a tread mill for ninety minutes without consuming any water. At thirty minute time points, each subject was weighed, and each subject's saliva was tested again for IgA and Amylase concentrations. Each subject's urine specific gravity was measured before and at the conclusion of the subject's ninety minute exercise period.

FIG. 10 shows the weight loss of one selected subject over the experimental study. The first three data points of FIG. 10 represent measurements taken on Days 1-3, and the fourth data point (100) corresponds to the sample taken on Day 4 prior to initiation of the subject's ninety minute exercise period. From the start to finish of the exercise period (corresponding to the fourth through seventh data points), this particular subject lost 0.8 kgs, of which nearly all is expected constitute water loss.

FIG. 11 shows the Amylase signal for the same selected subject over Days 1-4. The first three data points shown in FIG. 11 correspond to measurements taken on Days 1-3, and the fourth data point (101) corresponds to the sample taken on Day 4 prior to initiation of the subject's ninety minute exercise period. Relative to the fourth data point (101), the fifth through seventh data points represent an increasing signal, indicating an increased concentration of Amylase in saliva.

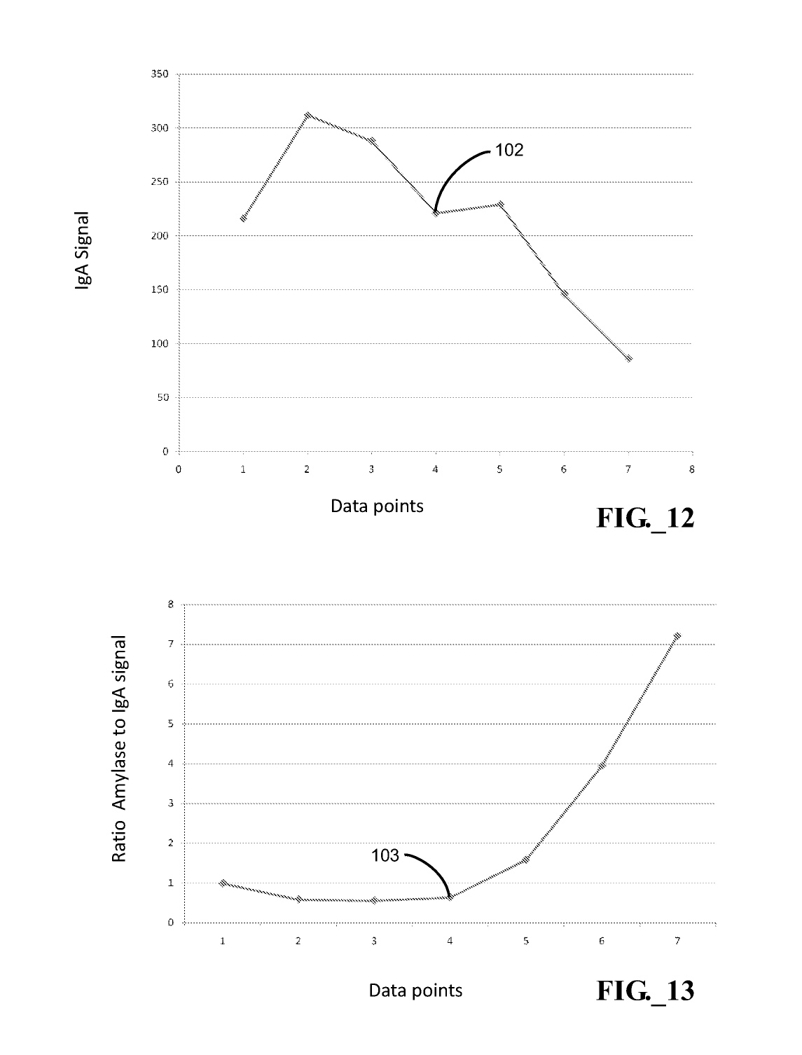

FIG. 12 shows the data for the IgA test strip over Days 1-4. The first three data points shown in FIG. 12 correspond to measurements taken on Days 1-3, and the fourth data point (102) corresponds to the sample taken on Day 4 prior to initiation of the subject's ninety minute exercise period. The fifth through seventh data points taken during the exercise periods (at times of 30, 60, and 90 minutes from the start of exercise, respectively) embody a decreasing trend in IgA signal in relation to the fourth data point (102) taken prior to initiation of the exercise period. Both FIGS. 11 and 12 embody some variation due to sampling and environmental changes in the subject's saliva, but the signal trends during the exercise period are clear.

FIG. 13 represents a plot of the ratio of Amylase signal to IgA signal. The first through fourth data points (taken prior to initiation of the subject's exercise period) represent baseline salivary Amylase/IgA ratios between about 0.7 to about 1.0. A salivary Amylase/IgA ratio of 2.0 provides a possible threshold indicator for onset of dehydration. What is noteworthy from FIG. 13 is the substantial change in signal ratio during the exercise period relative to the baseline ratios. This subject lost 1.3% of total body weight over the course of the study, of which the majority is assumed to be water loss. Such percentage of water loss would be considered mild dehydration by most people familiar with the art. Yet, despite this relatively small water loss, the ratio of salivary Amylase/IgA signal from start to finish of the exercise period (with the start corresponding to fourth data point (103) went up by approximately a factor of ten—i.e., from a ratio of about 0.7 to a ratio of about 7.

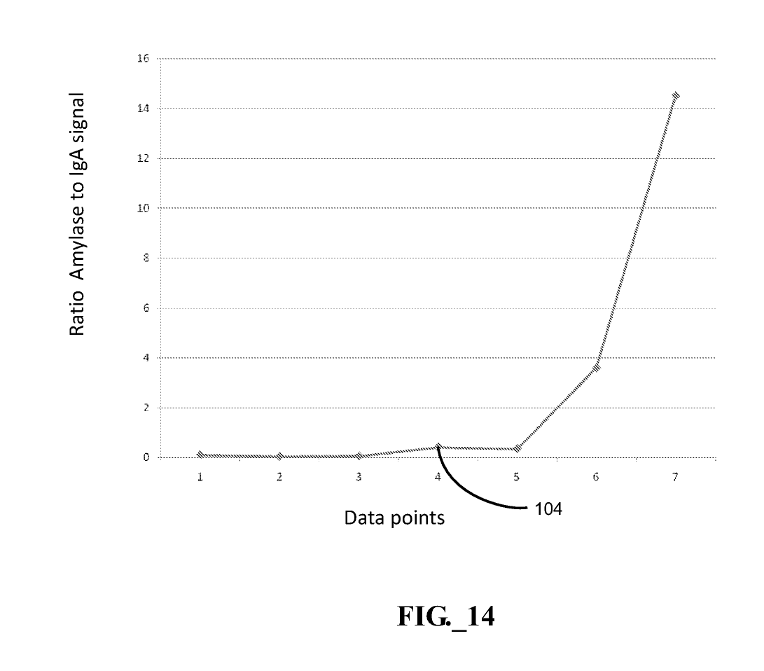

FIG. 14 shows the Amylase/IgA signals from the saliva of another subject of the four-day study, wherein the subject lost 2.4% body weight over the course of the experiment. This subject's Amylase/IgA signal ratio went up a factor of 34 over the exercise portion of the experiment (with the start of the exercise portion corresponding to the fourth data point (104)).

In a preferred embodiment, each assay for IgA and Amylase may be sensitized so that lines appear to be the same intensity for the majority of a patient population when in a hydrated state. Thus, upon testing of saliva of subjects who are not dehydrated, both lines would appear similar in intensity. A product may be set up in a format wherein two different test strips are placed side-by-side in a cartridge format that samples the saliva and automatically delivers the sample to the strips for analysis, to allow a patient to visually compare the two different test regions by eye. The product may have an indicator (such as an arrow or box around the Amylase test strip) indicating which line intensity should be considered as the marker for dehydration. When a patient is dehydrated, the indicated strip would be darker than the non-indicated strip. The greater the intensity changes between the two strips or test areas, the greater the level of dehydration would be indicated.

Alternatively, in one embodiment, both assays may be built into a single strip and co-located a small distance apart. In this case, the Amylase region may be marked in some fashion within a cartridge that samples and delivers the saliva. As before, the patient (or test provider) would visually inspect the two lines and if the indicated line is darker than the other line, then hydration would be diagnosed. The more intense the Amylase line relative to the IgA line, the greater the degree of dehydration would be indicated.

In another embodiment, a reader may be utilized to provide a superior level of hydration quantization. In this embodiment, a user (such as an elite athlete) could provide a saliva sample to a testing device prior to the onset of a training session. A reader may be utilized to sense and quantify the ratio of Amylase signal to IgA signal. As the athlete trains, the athlete will lose water through perspiration, respiration, etc. As the athlete rehydrates using liquid (e.g., oral liquid intake), the athlete's ratio of Amylase/IgA signal may be retested to monitor hydration status. In this manner, optimal liquid intake to maintain proper hydration may be determined without over-hydrating, which can also cause problematic health issues, and also impede athletic performance.

Similarly, a product embodying a reader to quantitate Amylase/IgA ratio may be used by a health care provider, such as a nurse or aide in a nursing home or long-term care facility, to monitor daily hydration status of an elderly patient or other long-term care patient. The patient could be measured at a time when hydration is considered acceptable, such as after the patient has been properly hydrated using an IV (intravenous line) or after the patient's liquid intake has been monitored carefully. As the patient self-hydrates on a daily basis through liquid intake, the health care provider could periodically monitor the patient's saliva to determine Amylase/IgA signal ratio. This ratio may be compared to the baseline level automatically through software built into the reader, to signal a relative status of hydration to the health care provider. In certain circumstances, a health care provider may more closely monitor the liquid intake if the patient is mildly dehydrated. In other cases, the health care provider may determine the patient is severely dehydrated requiring additional medical care such as IV hydration. Other product uses and diagnostic and treatment methods can also be envisioned by one skilled in the art.

While IgA and Amylase have been specifically discussed in connection with the preceding several figures, it is to be appreciated that detectable markers in saliva other than IgA and/or Amylase could also be considered. Any two salivary markers that either go up or go down during dehydration could also be used.

In certain patient populations or dehydration scenarios, the selection of biomarkers and assay format must be carefully considered. For example, in the subjects studied in the preceding example, both IgA and Amylase concentration increased during acute dehydration. Acute dehydration in this context means relatively fast dehydration (over a span of 90 minutes). In this same population, it may be found that Amylase increases during chronic dehydration while IgA decreases during chronic dehydration. In this context, chronic dehydration would mean long term dehydration where hydration status might have an effect on long term biological function. Thus, if these two biomarkers are used, different testing product embodiments would be necessary or desirable to facilitate direct or ratiometric comparison required for those two scenarios. Other testing product embodiments as described previously herein may be utilized.

As another example, it may be found that for healthy adult athletes, a testing product that quantitates Amylase and IgA in an inverted format may provide an excellent marker for dehydration in both acute and chronic settings. However, this format may not be appropriate for infants if these biomarkers are not up and down regulated in the same manner during dehydration. In that patient population, different biomarkers may be required.

While the invention has been has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein. Various elements and steps disclosed separately herein may be aggregated in different combinations and permutations to provide additional advantage(s) as may be desirable for a particular end use or application. Any one or more features of the following claims may be combined with one or more features of other claims (whether or not expressed in multiple dependent form) unless otherwise stated herein. Correspondingly, the invention as hereinafter claimed is intended to be broadly construed and interpreted, as including all such variations, modifications and alternative embodiments, within its spirit and scope.

What is claimed is:

1. A method of sensing a state of hydration or dehydration of a mammalian subject using saliva provided by or obtained from the mammalian subject, the method comprising:
contacting at least a portion of the saliva with at least one first indicator or test region disposed in or on a diagnostic device, and responsively generating a first signal correlative of concentration of at least one analyte in the saliva;
contacting at least a portion of the saliva with at least one second indicator or test region disposed in or on the diagnostic device, and responsively generating a second signal correlative of salivary flow of the mammalian subject; and
utilizing the first signal and the second signal to assess state of hydration or dehydration of the mammalian subject.

2. The method of claim 1, wherein the at least one analyte comprises IgA.

3. The method of claim 1, wherein the at least one analyte comprises albumin.

4. The method of claim 1, wherein the at least one analyte comprises a plurality of analytes, including IgA and albumin.

5. The method of claim 1, wherein the at least one first indicator or test region comprises a plurality of first indicators or test regions.

6. The method of claim 1, wherein the plurality of indicators or test regions comprises indicators or test regions adapted to generate signals correlative of concentration of different analytes.

7. The method of claim 1, wherein the at least one second indicator or test region comprises a plurality of second indicators or test regions.

8. The method of claim 1, wherein the at least one second indicator or test region comprises at least one pH sensing region.

9. The method of claim 1, wherein utilizing the first signal and the second signal includes comparing the first signal and the second signal.

10. The method of claim 1, wherein the diagnostic device comprises at least one lateral flow test strip.

11. The method of claim 10, further comprising maintaining at least a portion of the diagnostic device in a mouth of the mammalian subject to obtain the saliva.

12. The method of claim 10, further comprising maintaining at least a portion of the diagnostic device in a mouth of a human subject to obtain the saliva while the human subject is exercising.

13. The method of claim 1, wherein the at least one first indicator or test region comprises antibodies arranged to interact with the at least one analyte.

14. The method of claim 13, wherein the diagnostic device comprises at least one colorimetric indicator that is bound to the analyte in proportion to concentration of the at least one analyte.

15. The method of claim 1, further comprising correlating or comparing at least one of the first signal and the second signal to at least one baseline value for at least one of the first signal and the second signal.

16. The method of claim 15, wherein the at least one baseline value is specific to a population of similar age or disease condition, of which the mammalian subject is a member.

17. The method of claim 15, wherein the at least one baseline value is specific to the mammalian subject based on a prior sensing or assessment of state of hydration or dehydration of the mammalian subject.

18. The method of claim 1, further comprising treating at least a portion of the saliva with a buffer.

19. A method of sensing a state of hydration or dehydration of a mammalian subject using saliva provided by or obtained from the mammalian subject, the method comprising:
contacting at least a portion of the saliva with at least one first indicator or test region disposed in or on a diagnostic device, and responsively generating a first signal correlative of concentration of IgA in the saliva;
contacting at least a portion of the saliva with at least one second indicator or test region disposed in or on a diagnostic device, and responsively generating a second signal correlative of concentration of albumin in the saliva; and
utilizing the first signal and the second signal to assess state of hydration or dehydration of the mammalian subject.

20. The method of claim 19, wherein the diagnostic device comprises a lateral flow test strip including the at least one first indicator or test region and including the at least one second indicator or test region.

21. The method of claim 19, wherein the diagnostic device comprises a first lateral flow test strip including the at least one first indicator or test region, and comprises a second lateral flow test strip including the at least one second indicator or test region.

22. The method of claim 19, wherein utilizing the first signal and the second signal includes comparing the first signal and the second signal.

23. The method of claim 19, wherein the diagnostic device comprises at least one lateral flow test strip.

24. The method of claim 23, further comprising maintaining at least a portion of the diagnostic device in a mouth of the mammalian subject to obtain the saliva.

25. The method of claim 23, further comprising maintaining at least a portion of the diagnostic device in a mouth of a human subject to obtain the saliva while the human subject is exercising.

26. The method of claim 19, wherein the at least one first indicator or test region comprises first antibodies arranged to interact with IgA, and the at least one second indicator or test region comprises second antibodies arranged to interact with albumin.

27. The method of claim 19, wherein the diagnostic device comprises a first colorimetric indicator that is bound to IgA in proportion to concentration of IgA, and comprises a second colorimetric indicator that is bound to albumin in proportion to concentration of albumin.

28. The method of claim 19, further comprising correlating or comparing at least one of the first signal and the second signal to at least one baseline value for at least one of the first signal and the second signal.
